(19) United States
(12) Patent Application Publication
(10) Pub. No.: US 2004/0101836 A1
(43) Pub. Date: May 27, 2004

(54) Compositions, methods, apparatus and products comprising a stathmin/oncoprotein 18 sequence for detecting and treating cancer

(76) Inventors: Nikhil C. Munshi, (US); Jing Yi Wang, (US)

Correspondence Address: GILBRETH & ASSOCIATES, P.C., P.O. BOX 2428, BELLAIRE, TX, 77402-2428, US

(21) Appl. No.: 10/005,869

(22) Filed: Dec. 3, 2001

(51) Int. Cl. C12Q 1/68; A61K 39/395; A61K 48/00

(52) U.S. Cl. 435/6; 424/155.1; 514/44

(57) ABSTRACT

Methods, apparatus, compositions, and products for screening and/or treating cancer in a patient are disclosed. Methods, apparatus, compositions, and products comprise a stathmin/oncoprotein 18 sequence. The sequence of the invention may be a single stranded nucleic acid,a double stranded nucleic acid, a polypeptided, an antibody, an oligonucelotide, or any combination thereof.

### BACKGROUND OF THE INVENTION

1. Field of the Invention

The present invention relates to products, compositions, methods and apparatus for identification of a cancer and/or pre-cancerous cellular changes. In another aspect, the present invention relates to products, compositions, methods and apparatus for identifying a cancer and/or pre-cancerous cellular changes in a patient. In even another aspect, the present invention relates to products, compositions, methods and apparatus comprising a stathmin/oncoprotein 18 sequence for identifying a cancer or pre-cancerous cellular change in a patient. In still another aspect, the present invention relates to products, compositions, methods and apparatus for treatment of cancers and pre-cancerous cellular changes. In yet another aspect, the present invention relates to products, compositions, methods and apparatus for treatment of a cancer or pre-cancerous cellular change in a patient. In even still another aspect, the present invention relates to products, compositions, methods and apparatus comprising a stathmin/oncoprotein 18 sequence for treatment of a cancer or pre-cancerous cellular change in a patient.

2. Description of the Related Art

It is well established that carcinogenic changes in a wide variety of animal and human tissues are associated with genetic mutations in those tissues. Much research has been devoted to identifying and studying the role of oncogenes, as well as tumor suppressor genes, in hopes of identifying both new markers and treaments for cancer. Identification of genetic targets for treating cancer has led to use of gene therapy techniques either alone or in combination with the more traditional methods of chemotherapy and radiotherapy.

As early as 1977, researchers succeeded in using antisense technology to inhibit DNA expression, at least in cell-free systems. To date, antisense, short strands of RNA or DNA, has been reported to show activity against some viral pathogens (human immunodeficiency virus in vitro and cytomegalovirus in vivo) and also some cancers.

For example, Ciardiello et al. (Oncogene 1994 January; 9(1):291-8) report inhibition of CRIPTO expression and tumorigenicity in human colon cancer cells by use of antisense CRIPTO RNA and antisense CRIPTO oligodeoxynucleotides. CRIPTO is an epidermal growth factor-related gene expressed in a majority of human colorectal tumors.

Normanno et al. (Int. J. Cancer 1995; 62:762-766) describe the inhibition of growth and transformation of a human colon carcinoma cell line by use of amphiregulin anti-sense oligodeoxynucleotides. Amphiregulin is a secreted haparin-binding growth factor that is structurally and functionally related to epidermal growth factor and transforming growth factor α.

Numerous oncoproteins have been isolated to date. One such oncoprotein associated with a wide range of cancers is oncoprotein 18 (Op18), also known as stathmin. Stathmin/Op18 has been reported to have an effect on microtubule dynamics and spindle assembly during mitosis. Microtubule polymerization during the cell cycle is regulated by the balance between microtubule-stabilizing and -destabilizing factors. A recent article by Andersen (Trends Cell Biol 2000 July; 10(7):261-267) reports that Op18 may function as a microtubule-destabilizing factor.

In spite of advancements in the art, there remains a need for apparatus, compositions, methods and products for identifying cancers and pre-cancerous cellular changes, which do not suffer from the disadvantages of the prior art.

There is another need in the art for apparatus, compositions, methods and products comprising a stathmin/oncoprotein 18 sequence for identifying a cancer or a pre-cancerous cellular changes in a patient, which do not suffer from the disadvantages of the prior art.

There is even another need in the art for apparatus, compositions, methods and products for treatment of cancer and/or pre-cancerous cellular changes, which do not suffer from the disadvantages of the prior art.

There is still another need in the art for apparatus, compositions, methods and products comprising a stathmin/oncoprotein 18 sequence for treatment of cancer and prevention of pre-cancerous cellular changes in a patient, which do not suffer from the disadvantages of the prior art.

These and other needs in the art will become apparent to those of skill in the art upon review of this specification, including its drawings, claims and appendix.

### SUMMARY OF THE INVENTION

It is an object of the present invention to provide for apparatus, compositions, methods and products for identifying cancers and pre-cancerous cellular changes.

It is another object of the present invention to provide for apparatus, compositions, products and methods comprising a stathmin/oncoprotein 18 sequence for identifying cancers and pre-cancerous cellular changes in a patient.

It is even another object of the present invention to provide for apparatus, compositions, products and methods for treatment of cancer and prevention of pre-cancerous cellular changes.

It is still another object of the present invention to provide for apparatus, compositions, products and methods comprising a stathmin/oncoprotein 18 sequence for treatment of cancer and/or pre-cancerous cellular changes in a patient.

These and other objects in the art will become apparent to those of skill in the art upon review of this specification, including its drawings, claims and appendix.

According to one embodiment of the present invention there is provided a method of screening a patient for a cancer. The method generally comprises performing an amplification technique on a sample from a biopsy taken from a patient. The sample comprises nucleic acid, and the amplification technique is directed to specific amplification of a portion of a stathmin/oncoprotein 18 sequence contained therein. The method further includes probing for the presence of a stathmin/oncoprotein 18 sequence in the amplified sequence using a stathmin/oncoprotein 18 specific probe.

According to another embodiment of the present invention there is provided a method of screening a patient for a cancer. The method generally comprises contacting cellular material together with a stathmin/oncoprotein 18 specific probe. The cellular material is generally extracted from a sample, such as a biopsy, taken from a patient. The cellular material may be any purified, partially purified, or non-purified cellular material such as, for example, deoxyribonucelic acid (DNA), ribonucleic acid (RNA), polypepetides, or a combination thereof. The cellular material may be purified, either paritally or wholly, using any of the methods well known in the art.

According to even another embodiment of the present invention there is provided a method of treating a patient. The method generally comprises administering a composition comprising an effective amount of an antisense stathmin/oncoprotein 18 DNA sequence to a patient. Preferably, the antisense stathmin/oncoprotein 18 DNA sequence is expressed from a viral expression vector, such as an adeno-associated vector.

According to still another embodiment of the present invention there is provided a method of treating a patient. The treatment method generally comprises administering an effective amount of a composition to a patient, wherein the composition comprises an agent that inhibits expression of a stathmin/oncoprotein 18 gene.

According to yet another embodiment of the present invention there is provided a method of treating a patient. The treatment method generally comprises administering an effective amount of a composition to a patient, wherein the composition comprises an agent that specifically inhibits a stathmin/oncoprotein 18 protein. Inhibition of a protein can be by any of the methods known in the art, such as, targeting with an antibody, inhibition of post-translation modification, inhibition of protein stability and half-life. A preferred agent for use in the treatment method of the present invention is an antibody specific for interaction with an epitope of a stathmin/oncoprotein 18 protein.

According to even still another embodiment of the present invention there is provided a method of treating a patient. The method comprises transfecting dendritic cells (DCs) into a patient, wherein the dendritic cells have been altered to stably produce a stathmin/oncoprotein 18 antigen. Preferably, an adeno-associated virus (AAV) that has been genetically manipulated to comprise a portion of a stathmin/oncoprotein 18 antigen-encoding gene is used to infect monocyte precursors which are then induced to differentiate into Dcs.

According to even yet another embodiment of the present invention there is provided for a kit for screening a patient for a cancer. Generally the kit comprises a probe that is specific for the detection of a stathmin/oncoprotein 18 gene or gene product. The stathmin/oncoprotein 18-specific probe may be a single-stranded olidonucleotide sequence, a double-stranded oligonucletide sequence, a polypeptide, or any combination thereof.

These and other embodiments of the present invention will become apparent to those of skill in the art upon review of this specification, including its drawings and claims.

### BRIEF DESCRIPTION OF THE DRAWINGS

FIG. 1 shows results of a differential screening of a human myeloma cDNA library using (A) a myeloma-specific probe, or (B) a normal plasma cell probe.

FIG. 2 is a Southern blot of differentially expressed cDNA clones isolated by differential screening.

FIG. 3 shows the expression of the stathmin/Op18 gene in myeloma cell lines.

FIG. 4 is an RT-PCR analysis of the expression of stathmin/Op18 mRNA in bone marrow CD138+ cells from myeloma patients.

FIG. 5 is a Southern blot analysis for stathmin/Op18 gene in human myeloma cell lines.

FIG. 6 shows fluorescein-labeled antisense stathmin/Op18 ODN uptake in human myeloma cells.

FIG. 7 shows dose-response effects of sense (closed circles) and antisense (open circles) stathmin/Op18 oligonucleotides on ARD myeloma cell number in liquid culture.

FIG. 8 shows growth inhibition of myeloma cells by specific stathmin/Op18 antisense oligonucleotides.

FIG. 9 is a Northern blot analysis of the expression of stathmin/Op18 gene in myeloma ARP cell line transected with stathmin/Op18 sense and antisense oligodeoxynucleotides (ODN).

FIG. 10 shows the results of an analysis of DNA synthesis in ARD cells treated by specific stathmin/Op18 antisense oligonucelotides.

FIG. 11 depicts a schematic of the pBAD-Thio-Stathmin/Op18 vector and shows proteins expressed from the vector.

FIG. 12 is a Western blot analysis showing prevalence of antibody against stathmin/Op18 in myeloma patients.

### DETAILED DESCRIPTION OF THE INVENTION

The present invention is directed to the use of a stathmin/oncoprotein 18 sequence for detecting cancer in a sample. The present invention is also directed to the use of a stathmin/oncoprotein 18 sequence for treating cancer in a patient afflicted with a cancer in any stage of development. The stathmin/oncoprotein 18 sequence utilized may be any of the stathmin sequences known in the art, such as those in Genbank. The cancer may be any cancer, such as, for example, lymphoma, myeloma, leukemia, breast, dermal, oral, colon, penile, vulvar, and any combination thereof. In a preferred embodiment the cancer is a myeloma, more preferably multiple myeloma.

The stathmin/oncoprotein 18 sequence of the invention may be any type of sequence, such as a single stranded nucleic acid, a double stranded nucleic acid, an oligonucelotide, a polypeptide, an antibody, or any combination thereof. The size and length of the sequence is not restricted.

The inventors of the present invention have discovered that the phosphoprotein stathmin/oncoprotein 18 gene is overexpressed in human myeloma cell lines as compared to tonsillar plasma cells and normal human fibroblasts. The inventors have also determined that, surprisingly, the growth rate of myeloma cells is inhibited as a result of treatment with antisense stathmin/oncoprotein 18 oligodeoxynucleotides.

According to one embodiment of the present invention there is provided a method of screening a patient for a cancer. The method generally comprises performing an amplification technique on a sample from a biopsy taken from a patient. The sample comprises cellular material, preferably nucleic acid, and the amplification technique is directed to specific amplification of a portion of a marker for a cancer. In a preferred embodiment, the marker is a stathmin/oncoprotein 18 sequence. The method allows for determining whether the stathmin/oncoprotein 18 sequence is overexpressed. The cancer may be any cancer, such as, for example, lymphoma, leukemia, myeloma, breast, dermal, oral, colon, penile, vulvar cancer, or any combination thereof. The cancer may be in any stage of development. The method may further include probing for the presence of a stathmin/oncoprotein 18 sequence in the amplified sequence using a stathmin/oncoprotein 18 specific probe. The probes used in the present invention may be of any size useful for the intended purpose and may be complementary to any portion of the stathmin/oncoprotein gene. Methods and strategies for design, construction, and purification of probes are well known by one of ordinary skill in the art, and all such methods and strategies are incorporated and may be used herein. The stathmin/oncoprotein 18 gene sequences are known and may be easily obtained from GenBank, as known by one of ordinary skill in the art.

Preparation of samples for amplification is well known in the art, and any such technique may be used herein. Amplification methods are well known in the art and include techniques such as, for example, polymerase chain reaction (PCR) amplification and reverse transcription PCR (RT-PCR), as well as others. The amplified products may be detected and analyzed using any of the numerous techniques well known in the art.

Another embodiment of the present invention provides for a method of screening a patient for a cancer. The method generally comprises contacting cellular material together with a stathmin/oncoprotein 18 specific probe. The cellular material is generally extracted from a sample, such as a biopsy, taken from a patient. The cellular material may be any purified or non-purified cellular material such as, for example, deoxyribonucelic acid (DNA), ribonucleic acid (RNA), polypepetides, or a combination thereof. The cellular material may be purified, either partially or wholly, using any of the purification methods well known in the art.

The stathmin/oncoprotein 18 probes useful in the screening methods of the present invention may be any type of probe known in the art useful in detecting the presence of stathmin/oncoprotein 18. These probe types include, but are not limited to, a single-stranded or double-stranded oligonucleotide sequence complementary to at least a portion of the plus or minus strand of a stathmin/oncoprotein 18 DNA sequence, a single-stranded or double-stranded oligonucleotide sequence complementary to at least a portion of a stathmin/oncoprotein 18 mRNA sequence, and an antibody specific to at least one epitope of a stathmin/oncoprotein 18 protein.

Numerous methods of detecting targeted sequences by use of a probe are well known in the art and all such methods are included herein. All hybridization methods known in the art are also suitable for use in the present invention.

The screening methods of the present invention may be performed on any organism capable of developing cancer. Preferably, the method of the present invention is performed on mammals, more preferably a human. The screening methods described herein are useful in detecting numerous types of cancer, such as, for example, lymphoma, leukemia, myeloma, breast, dermal, oral, colon, penile, vulvar cancer, and any combination thereof. In addition, the screening methods of the present invention are useful in detecting a cancer in any stage of development.

Even another embodiment of the present invention provides for a method of treating a patient. The method generally comprises administering a composition comprising an effective amount of an antisense stathmin/oncoprotein 18 sequence to a patient. The size of the sequence is not limited and can range in size from that of an oligonucleotide to that of a transcript. The antisense stathmin/oncoprotein 18 sequence may comprise DNA, RNA, ribosomal RNA, or any combination thereof.

The stathmin/oncoprotein 18 sequence may be expressed from any recombinant expression vector known in the art. Suitable vectors known in the art include, for example, mammalian expression vectors and viral vectors. Examples of viral vectors suitable for use in the present invention include: retroviruses; adenoviruses; adenoviral/retroviral chimeras; adeno-associated viruses; herpes simples virus I or II; parvovirus; and reticuloendotheliosis virus. Other possible viral vectors may be derived from poliovirus, papillomavirus, vaccinia virus, lentivirus, as well as chineric vetors incorportation favorable aspects of any two or more of the the above viruses. Preferably, the antisense stathmin/oncoptrotein 18 sequence is expressed from a recombinant viral expression vector, such as an adeno-associated vector.

Still another embodiment of the present invention provides for a method of treating a patient. The treatment method generally comprises administering an effective amount of a composition to a patient, wherein the composition comprises an agent that inhibits expression of the stathmin/oncoprotein 18 gene.

The numerous mechanisms for inhibiting the expression of a gene are well known in the art and include, but are not limited to, inhibiting gene transcription, inhibiting the messenger RNA (mRNA) of a gene via antisense technology, inhibiting translation of mRNA, inhibiting post-translational modification of a gene product, and inhibiting a gene product via immunotherapy techniques. These inhibition methods may be direct or indirect. Any one or combination of these mechanisms may be used in the present invention.

Suitable agents for use in the treatment methods of the present invention include, a nucleic acid sequence comprising antisense stathmin/oncoprotein 18 DNA, RNA or ribosomal RNA, and a nucleic acid sequence comprising DNA, RNA, or ribosomal RNA comprising a sequence complementary to the plus or the minus strand of stathmin/oncoprotein 18 DNA.

Administration of compositions of the present invention to a recipient may be by any method known in the art. Thus, administration of the present invention to a recipient may be by a route selected from oral, parenteral (including, subcutaneous, intradermal, intramuscular, and intravenous) and rectal. For increased efficacy, the compositions of the present invention may be administered via localized delivery to the targeted tissue, such as, for example, breast tissue in the case of breast cancer.

Yet another embodiment of the present invention provides for a method of treating a patient. The treatment method generally comprises administering an effective amount of a composition to a patient, wherein the composition comprises an agent that specifically inhibits the stathmin/oncoprotein 18 protein. Inhibition of a protein by immunotherapy techniques can be by any of at least one or any combination of the methods known in the art, such as, inhibition of gene expression, targeting a protein with an antibody, inhibition of post-translation modification, decrease of protein stability and decrease in amount of time of protein half-life. Immunotherapy suitable for the present invention may also include any combination of these known techniques. A preferred agent for use in the treatment method of the present invention is an antibody specific for interaction with an epitope of the stathmin/oncoprotein 18 protein.

Additional inhibitory agents suitable for use in the compositions and methods of the invention include agents wherein the agent is a DNA, cDNA, RNA, ribosomal RNA, or polypeptide sequence. Suitable examples of such agents include, an antisense stathmin/oncoprotein 18 sequence which inhibits transcription or translation of the stathmin gene or gene product, transcription factors which decrease expression of the stathmin gene, factors which affect translation of stathmin mRNA, factors which decrease the stability/half-life of stathmin mRNA, factors which decrease the stability/half-life of a stathmin polypeptide, and factors which interact with a stathmin polypeptide, such as a polypeptide encoding an antibody which specifically interacts with an epitope of oncoprotein 18. The material and methods for producing these types of inhibitors (DNA, cDNA, RNA and polypeptide) are known in the art and are included in the present invention.

For example, expression vectors encoding at least a portion of a negative regulator of stathmin gene transcription, or expressing a sequence inhibitory to translation of stathmin mRNA are within the scope of stathmin inhibitors defined herein. Expression vectors suitable for the present invention may encode for an antisense stathmin sequence, or at least a portion of a negative regulator of stathmin gene transcription.

Even still another embodiment of the present invention provides for a method of treating a patient. The method generally comprises transfecting dendritic cells (DCs), primed T cells, or a combination thereof, into a patient, wherein the dendritic cells have been altered to stably produce a stathmin/oncoprotein 18 antigen. The basis for using DC cells for human immunotherapy has recently been established as described in Butch A W, Kelly K A, Munshi N C. Dendritic cells derived from multiple myeloma patients efficiently internalize different classes of myeloma protein. Exp Hematol. 2001 January; 29(1):85-92; Santin, A. D., Hermonat, P. L., Ravaggi, Al, Chiriva-Internati, M., Hiserotdt, J. C., Pecorelli, S., and Parham, G. P. (1999) Kinetics of expression of surface antigens during the differentiation of human dendritic cells versus macrophages. Immunobiology 2000; and Young, J. W., and Inaba, K. (1996) DCs as adjuvants for class I manor histocompatibility complex-restricted antitumor immunity. J. Exp. Med. 183:7-11, all of which are incorporated herein by reference.

Specifically, a recombinant viral vector that has been genetically manipulated to comprise a portion of a stathmin/oncoprotein 18 gene is used to infect monocyte precursors acquired from a blood sample of a patient. The monocytes are induced to differentiate into DCs by treatment with a cytotkine. Examples of suitable cytokines include, but are not limited to IL-4, other interluekins, GM-CSF, TNF, INF, and any combination thereof. The dendritic cells may also stably produce a cytokine from a recombinant vetor.

Techniques for transferring genes are well known in the art, and any of those techniques may be used to produce dendritic cells that stably produce the antigen of choice. Preferably, an adeno-associated virus (AAV) that has been genetically manipulated to comprise a portion of an oncoprotein 18 antigen-encoding gene is used to infect monocyte precursors which are then induced to differentiate into DCs.

The treatment methods of the present invention may be performed on any organism having a cancer. Preferably, the methods of the present invention are performed on a human. The treatment methods described herein may be useful in treating numerous types of cancer, such as, for example, lymphoma, leukemia, myeloma, breast, dermal, oral, colon, penile, vulvar cancer, and any combination thereof. In addition, the treatment methods of the present invention may be useful against cancer in any stage of development.

The compositions and methods of the present invention are suitable for use with any organism afflicted with any stage of a cancer. Suitable organisms include mammals such as, humans, dogs, cats, horses, cows, sheep, goats, pigs, rats and mice. As mentioned, preferably the patient is human. The compositions and methods of the present invention are also suitable for use in any tissue or cell line that serves as a model for the study of cancer. Thus, the present invention is useful to numerous medical and health care professionals including, research scientists, medical doctors and veterinarians.

It should be noted that the present invention encompasses any and all methods for screening a patient for a cancer or a pre-cancerous cellular change, wherein the method comprises detection of stathmin/oncoprotein 18 expression. Any and all methods for treating a patient having a cancer, wherein the method comprises inhibition of a stathmin/oncoptrotein 18 gene or gene product are also well within the scope of the invention.

The compositions useful in the methods of the present invention further comprise a pharmaceutically acceptable carrier/vehicle. Pharmaceutically acceptable carriers/vehicles are known in the art and include aqueous solutions, non-toxic excipients, including salts, preservatives, buffers and the like, propylene glycol, polyethylene glycol, vegetable oil, injectable organic esters such as ethyloleate, water, saline solutions, parenteral vehicles such as sodium chloride and Ringer's dextrose, glycerol, lipids, alcohols.

Compositions of the present invention may be in any form known in the art, such as an orally digestible form, a sterile injectable form, forms suitable for delayed release, and forms that are enterically coated. Compositions of the invention may be in solid forms, including, for example, powders, tablets, pills, granules, capsules, sachets and suppositories, or may be in liquid forms including solutions, suspensions, gels and emulsions.

The compositions and methods of the present invention may be administered to a recipient/patient as a single dose unit, or may be administered in several dose units, for a period ranging from one day to several years. The dose schedule is dependent upon at least the severity of the cancer, as well as the mode of administration. The effective dose of the compositions of the present invention is further dependent upon the body weight (BW) of the recipient/patient and also upon the chosen inhibitor. Generally the compositions of the present invention are administered orally or intravenously.

Even still another embodiment of the present invention provides for a kit for screening a patient for a cancer or a pre-cancerous cellular change. Generally the kit comprises a probe that is specific for the detection of stathmin/oncoprotein 18. The stathmin-specific probe may be a single-stranded oligonucleotide sequence, a double-stranded oligonucletide sequence, a polypeptide, or any combination thereof.

The kit of the present invention is useful in screening any organism capable of developing a cancer. Preferably the patient is human. The kit of the present invention may be useful in detecting a cancer that is in any stage of development, and may be useful in detecting any cancer, such as, for example lymphoma, leukemia, myeloma, breast, dermal, oral, colon, penile, vulvar cancer, and any combination thereof.

Even yet another embodiment of the present invention provides a composition for treating a patient having a cancer. Generally, the composition comprises an effective amount of a stathmin sequence. Preferably, the effective dose utilized is of a concentration high enough to maximally inhibit growth of cancer cells, yet low enough to minimize negative impact on the growth of normal cells. The effective dose may be dependent upon factors such as body weight of patient, health of the patient, and additional therapies being administered to the patient.

The size of the stathmin/oncoprotein 18 sequence utilized in the present invention is not limited. The stathmin sequence of the composition may comprise single-stranded nucleic acids, double-stranded nucleic acids, polypeptides, and any combination thereof.

All references cited herein, including research articles, all U.S. and foreign patents and patent applications, are specifically and entirely incorporated by reference.

TABLE 1
S [symbol] mary of Mr [symbol] cDNA Clorres Ce [symbol] ed from [symbol] Mr [symbol] a Patient
CDNA Library by D [symbol] crential Screerring

| | | | Differential |
|---|---|---|---|
| [symbol] | Size (kb)^{b} | G [symbol] ^{c} | Expression [symbol] |
| 1 | 0.45 | Micro [symbol] nal signal p [symbol] c complex | 11-fold |
| | | 75 KD Submit O15005^{d} | |
| 9 | 1.0 | Op18 (Also known as o [symbol] coptotein18, | 9-fold |
| | | st [symbol] ctc.) [symbol] 04991 | |
| 1 [symbol] | 1.0 | A [symbol] ribo [symbol] tol ph [symbol] ph [symbol] tein [symbol] .X15096 | 13-fold |
| 18 | 1.1 | Mnt [symbol] [symbol] ide [symbol] fale [symbol] | 14-fold |
| | | protein (SAP-MU-o) M60257 | |
| 27 | 0.9 | Nuclcer protein ty [symbol] phosph [symbol] se, U4 [symbol] 2 [symbol] 6 | 6-fold |
| 29 | 0.5 | Unknow. U88153 | 8-fold |
| 33 | 0.6 | N [symbol] l [symbol] r chlorideion ch [symbol] el protein | 21-fold |
| | | (NCC [symbol] ) [symbol] , [symbol] 9 [symbol] 05 | |
| 38 | 0.75 | Tr [symbol] n [symbol] (TACLN2). [symbol] M003564 1 | 8-fold |
| 1 | 0.45 | Micro [symbol] l signal peptid [symbol] complex | 11-fold |
| | | 25 KD Submit. | |
| 9 | 1.0 | Op18 (Also known as oncopro [symbol] 18. | 9-fold |
| | | st [symbol] cot.) | |
| 10 | 1.0 | A [symbol] c nbosot [symbol] l p [symbol] osphp [symbol] in PO | 13-fold |
| 18 | 1.1 | [symbol] c [symbol] s [symbol] [symbol] tor | 14-fold |
| | | protein (SAP-MU-O) | |
| 27 | 0.9 | Nucl [symbol] r protein ty [symbol] pbo [symbol] | 6-fold |
| 29 | 0.5 | Unknow. | 8-fold |
| 33 | 0.6 | Nuel [symbol] [symbol] deion channel protein, | 21-fold |
| | | (NCC27) [symbol] . | |
| 38 | 0.75 | Traregehn 2 (TACLN2). | 8-fold |

### EXAMPLES

The following examples are provided to illustrate the present invention. These examples are not intended to limit the scope of the claims of the present invention, and should not be so interpreted.

### Example 1

Differential screening of a myeloma cDNa library

Shown in FIG. 1 are results from a differential screening analysis of a myeloma cDNA Library. After in vivo mass excision of phagemids, cDNA library in E. coli was plated onto LB agar plates. Plates were incubated at 37° C. for 18 hours and then blotted onto Hybond™-C extra nitrocellulose membranes. For each Petri dish, two transfers were performed. One filter was hybridized with the myeloma-specific probe (A), the second one with normal plasma cell probe (B). Clones giving differential hybridization signals ( [symbol] ) were selected. Clones encoding human immunoglobulin sequences were subsequently eliminated during the secondary screening with IgGγ and K probes (ATCC, Cat. 61229 and 59173).

### Example 2

Southern blot analysis of cDNA clones differentially expressed in myeloma versus normal cells.

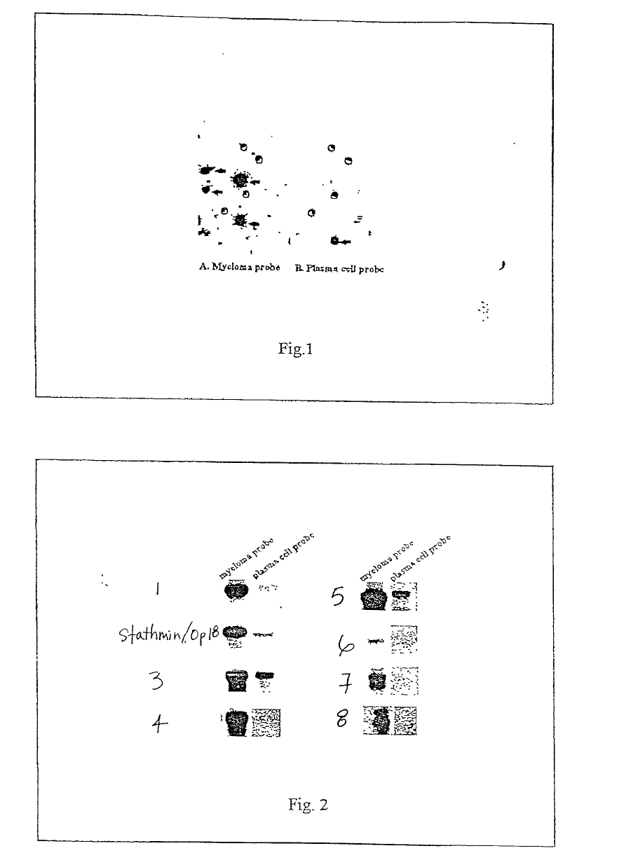

Shown in FIG. 2 is a southern blot analysis of differentially expressed cDNA clones as identified in Table 1. PCR was performed to amplify eight different cDNA inserts. The PCR-amplified products were electrophoresed in 0.8% agarose gel, transferred to nylon membrane and hybridized with subtracted normal plasma cell-probe first, then the same blot was subsequently stripped and hybridized with subtracted myeloma-probe.

### Example 3

Expression of Stathmin/Oncoprotein 18 in myeloma cells.

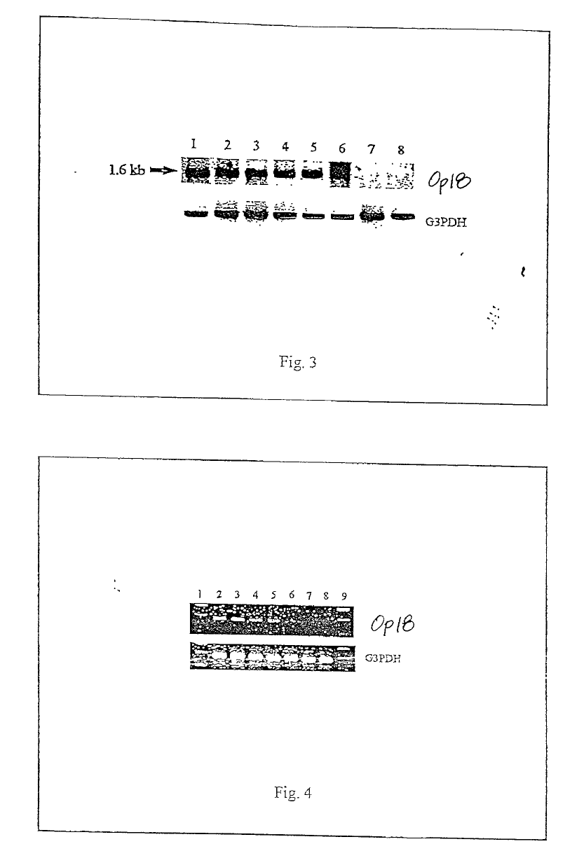

FIG. 3 illustrates results of expression of stathmin/Op18 gene in myeloma cell lines. Twenty (20) μg of total RNA from each cell line were size-fractionated on a denaturing formaldehyde agarose gel, blotted onto nylon filters (Hybond-N; Amersham), and probed with a stathmin/Op18 cDNA probe. RNA was extracted from the following sources: Lane 1, U266; Lane 2, ARD; Lane 3, ARP; Lane 4, ARK; Lane 5,RPMI8226; Lane 6, K562; Lane 7, tonsil CD138⁺ cells; Lane 8, normal human fibroblasts. A probe of G3PDH was used as a control to ensure uniform RNA loading.

### Example 4

RT-PCR analysis of stathmin/Op18 mRNA expression in bone marrow CD138⁺ cells from myeloma patients.

Given in FIG. 4 are results of an RT-PCR analysis of the expression of stathmin/Op18 mRNA in bone marrow CD138⁺ cells from myeloma patients. cDNA from 4 myeloma patient samples and 3 normal control were amplified by RT-PCR with specific primers. G3PDH was amplified as an internal control to ensure uniform RNA loading. The sources of RNA used in the numbered lanes of this Figure were: Lanes 2-5, patient bone marrow CD138⁺ cells; Lane 6, normal bone marrow MNC; Lane 7, tonsil CD138⁺ cells; Lane 8, normal human fibroblasts; and Lanes 1 and 9, molecular weight marker.

### Example 5

Southern blot analysis of Stathmin/Oncoprotein 18 in human myeloma cell lines.

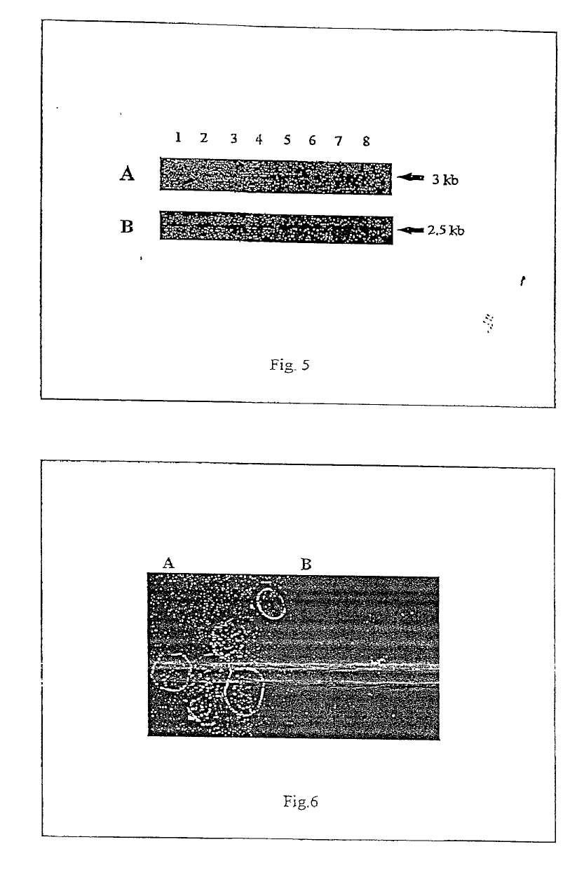

The results of a southern blot analysis for the stathmin/Op18 gene in human myeloma cell lines are provided in FIG. 5. Twenty μg of DNA were digested with EcoRI (A), or HindIII (B), and hybridized with a cDNA for Op18. The sources of DNA were: Lane 1, ARD; Lane 2, ARP; Lane 3, ARH-77; Lane 4, ARK; Lane 5, U266; Lane 6, RPMI 8226; Lane 7, tonsil MNC; Lane 8, normal human fibroblasts.

### Example 6

Use of antisense stathmin/oncoprotein 18 loligodeoxynucleotides in human myeloma cells.

Fluorescein-labeled stathmin/Op18 antisense ODN uptake in human myeloma cells are shown in FIG. 6. Phase-contrast (A) and fluorescence (B) confocal photomicrographs of human myeloma cells ARP that were transfected with flourescein-labeled ODN (5 μmol/L) for one day are shown. Cells were fixed with 5% paraformaldehyde and examined by a laser scanning confocal microscope.

### Example 7

Effects of antisense stathmin/oncoprotein 18 loligodeoxynucleotides in human myeloma cells.

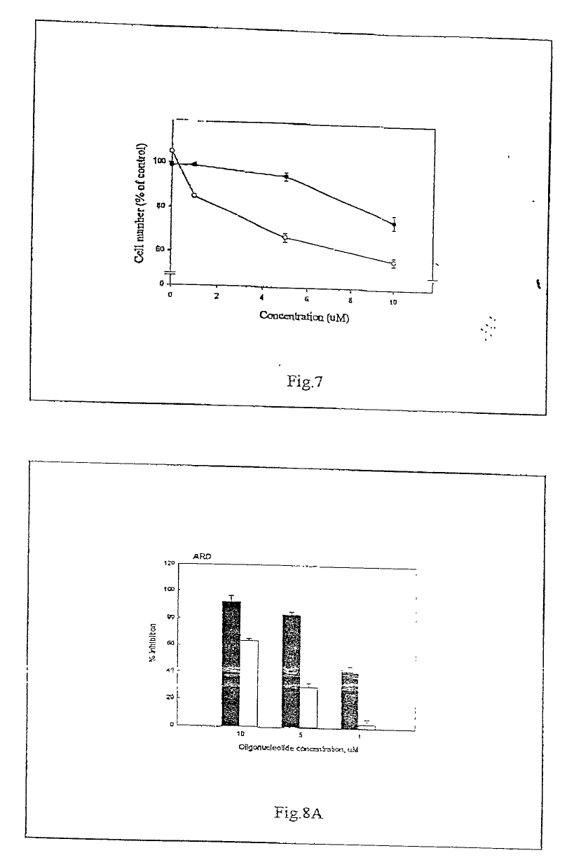

FIG. 7 is a graph illustrating dose-response effects of sense (●) and antisense (◯) stathmin/Op18 oligonucleotides on ARD myeloma cell number in liquid culture. Cells were exposed for 4 days after which time they were counted. Results are expressed as a mean percentage (±S.E.) of control with no oligonucleotide. Each point represents the mean of at least three experiments with duplicate wells.

### Example 8

Inhibition of ARD myeloma cell growth by specific stathmin/Op18 antisense oligonucleotides.

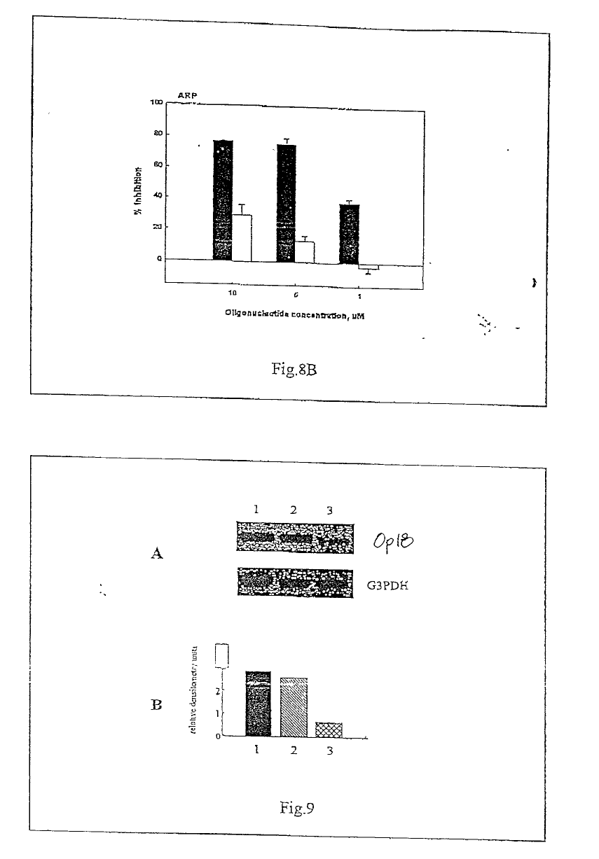

FIG. 8A is a bar graph of ARD myeloma cell growth inhibited by specific stathmin/Op18 antisense oligonucleotides. Myeloma cell growth inhibition is measured by of the amount of [³H] thymidine incorporation by the cells in the presence of antisense oligomers. The data presented are means of three experiments ±SD. Closed bars (▪) are antisense treated cultures. Open bars ( [symbol] ) are control sense-treated cultures.

FIG. 8B is a bar graph of ARP myeloma cell growth inhibited by specific stathmin/Op18 antisense oligonucleotides. Myeloma cell growth inhibition is measured by of the amount of [³H] thymidine incorporation by the cells in the presence of antisense oligomers. The data presented represent means of three experiments ±SD. Closed bars (▪) are antisense treated cultures. Open bars ( [symbol] ) are control sense-treated cultures.

### Example 9

Northern blot analysis of stathmin/oncoprotein 18 gene in myeloma ARP cell line transfected with sense or antisense stathmin/Op18 oligodeoxynucleotides.

Provided in FIG. 9A is a northern blot analysis of the expression of stathmin/Op18 gene in a myeloma ARP cell line transfected with stathmin/Op18 sense and stathmin/Op18 antisense ODN (5 μmol/L each) for three days. Twenty μg of total RNA were size-fractionated on a denaturing formaldehyde agarose gel, blotted onto nylon filters (Hybond-N; Amersham), and probed with a stathmin/Op18 cDNA. Sources of RNAs were: Lane 1, ARP cells with no added oligonucleotide; Lane 2, ARP cells transfected with stathmin/Op18 sense oligonucleotide.

FIG. 9B is a bar graph showing the relative amount of stathmin/Op18 mRNA in the myeloma cells transfected with sense and antisense oligonucleotide by densitometric analysis. Lane 1 shows ARP cells with no added oligonucleotide; Lane 2 shows ARP cells transfected with stathmin/Op18 sense oligonucleotide; and Lane 3 shows ARP cells transfected with stathmin/op18 antisense oligonucleotide.

### Example 10

DNA synthesis in ARD cells treated by specific stathmin/Op18 antisense oligonucleotides.

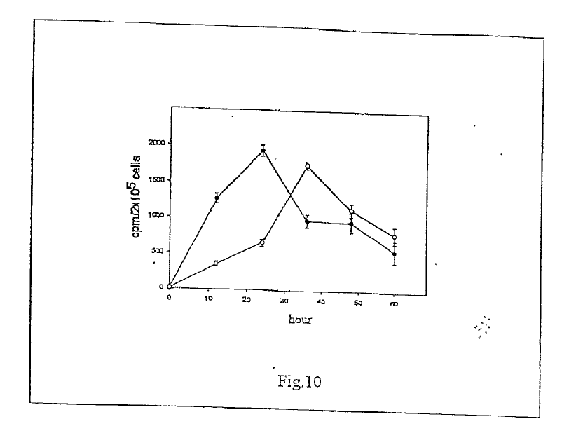

FIG. 10 is a graph depicting DNA synthesis, measured by incorporation of [³H] thymidine, in ARD cells treated by specific stathmin/Op18 antisense oligonucleotides, [³H] thymidine was measured as cpm/2×10⁵ cells versus time. Cells were starved for 24 hours, treated with sense (S) (●) or antisense (AS) ODN (◯), and subjected to incorporation of [³H] thymidine. At the time points indicated in FIG. 10, the amount of [³H] thymidine incorporation was determined.

### Example 11

Cloning and Expression of Stathmin/Op18

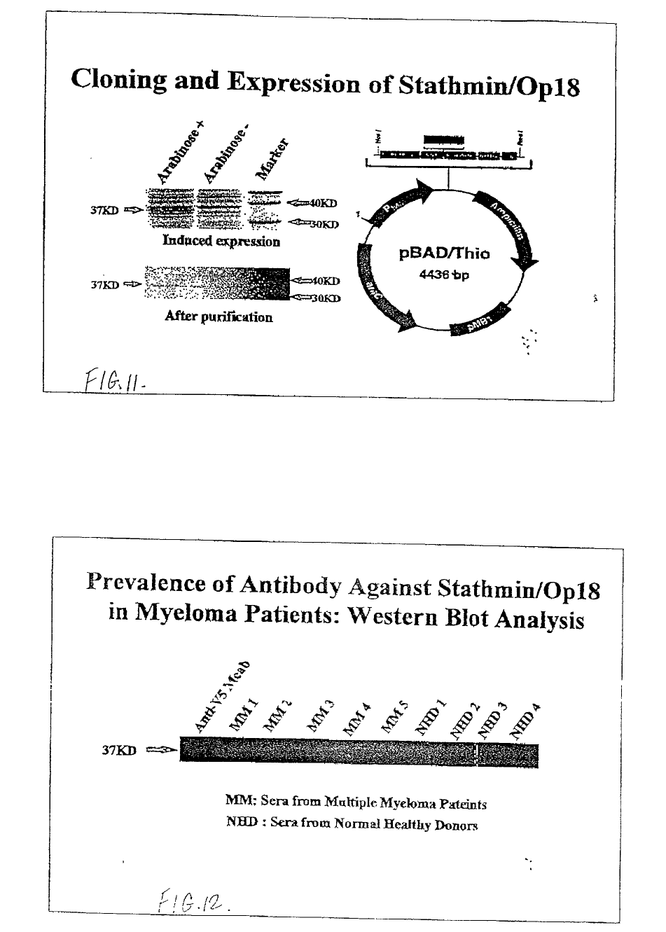

A stathmin/oncoprotein 18 fragment was sub-cloned into the arabinose-inducible expression vector pBAD-Thio to produce the pBAD-Thio-Stathmin vector. As shown in FIG. 11, high levels of expression of a 37 kD stathmin/oncoprotein 18 protein from the pBAD-Thio-Stathmin vector is dependent upon treatment with arabinose. The 37 kD stathmin/oncoprotein 18 protein is a stable protein following purification as shown in the lower left panel of FIG. 11.

### Example 12

Western Blot Analysis of Stathmin/Oncoprotein 18 in Myeloma Patients.

The purified 37 kD stathmin protein expressed from the pBAD-Thio-Stathmin/Oncoprotein arabinose-inducible vector was used to evaluate immune response in myelom patients. As shown in FIG. 12, purified IgG from plasma derived from multiple myeloma patients form an immune complex with the purified stathmin protein, see lanes labeled MM1-MM5. In contrast, IgG purified from plasma from healthy donors did not show a similar response.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein but rather that the claims be construed as encompassing all the features of patentable novelty which reside in the present invention, including all features which would be treated as equivalents thereof by those skilled in the art to which this invention pertains.

All references cited in the present application, including journal articles, U.S. Patents, and patent applications, are herein incorporated by reference.

We claim:

1. A method of screening a patient for cancer, the method comprising:
a) performing an amplification technique on a sample from a biopsy taken from a patient to produce an amplified sample, wherein the sample comprises nucleic acid, and wherein the amplification technique is specific for amplification of a portion of a stathmin/Op18 mRNA sequence.

2. The method of claim 1 wherein the patient is a human, wherein the cancer is in any stage of development, and wherein the cancer is selected from the group consisting of lymphoma, leukemia, myeloma, breast, dermal, oral, colon, penile, vulvar cancer, and any combination thereof.

3. The method of claim 1 wherein the amplification technique is polymerase chain reaction amplification.

4. The method of claim 1 wherein the amplification technique is reverse-transcription polymerase chain reaction amplification.

5. A method of screening a patient for a cancer, the method comprising:
a) contacting cellular material with a stathmin/Op18 specific probe, wherein the cellular material is extracted from a biopsy taken from a patient.

6. The method of claim 5 wherein the cellular material comprises nucleic acid, polypepetides, or a combination thereof.

7. The method of claim 5 wherein the probe is a stathmin/Op18 DNA or RNA oligonucleotide sequence complementary to the plus strand of a stathmin/Op18 DNA sequence.

8. The method of claim 5 wherein the probe is a stathmin/Op18 DNA or RNA oligonucleotide sequence complementary to a portion of a stathmin/Op18 mRNA sequence.

9. The method of claim 5 wherein the probe is a stathmin/Op18 DNA or RNA oligonucleotide sequence complementary to a portion of a stathmin/Op18 ribosomal RNA sequence.

10. The method of claim 5 wherein the probe is an antibody specific to an epitope of a stathmin/Op18 protein.

11. The method of claim 5 wherein the cancer is in any stage of development, and wherein the cancer is selected from the group consisting of lymphoma, leukemia, myeloma, breast, dermal, oral, colon, penile, vulvar cancer, and any combination thereof.

12. A method of treating a patient comprising:
a) administering a composition comprising an effective amount of an antisense stathmin/Op18 sequence to a patient.

13. The method of claim 12 wherein the antisense stathmin/Op18 sequence is expressed from a viral expression vector.

14. The method of claim 13 wherein the patient is human and has a cancer in any stage of development.

15. The method of claim 14 wherein the cancer is lymphoma, leukemia, myeloma, breast, dermal, oral, colon, penile, vulvar cancer, or any combination thereof.

16. A method of treating a patient comprising:
a) administering an effective amount of a composition to a patient, wherein the composition comprises an agent that inhibits expression of a stathmin/Op18 gene.

17. The method of claim 16 wherein the agent is an oligonucleotide comprising antisense stathmin/Op18 DNA, RNA or ribosomal RNA.

18. The method of claim 17 wherein the agent is an oligonucleotide comprising sequences complementary to the plus or minus strand of stathmin/Op18 DNA.

19. The method of claim 18 wherein the patient is human and has a cancer in any stage of development.

20. The method of claim 19 wherein the cancer is lymphoma, leukemia, myeloma, breast, dermal, oral, colon, penile, vulvar cancer, or any combination thereof.

21. A method of treating a patient comprising:
a) administering an effective amount of a composition comprising an agent that specifically inhibits a stathmin/Op18 protein.

22. The method of claim 21 wherein the agent is an antibody specific for a stathmin/Op18 protein.

23. The method of claim 22 wherein the patient is human and has a cancer in any stage of development.

24. The method of claim 23 wherein the cancer is lymphoma, leukemia, myeloma, breast, dermal, oral, colon, penile, vulvar cancer, or any combination thereof.

25. A method of treating a patient comprising:
a) transfecting dendritic precursor cells of a patient with a recombinant viral vector wherein said vector drives expression of a stathmin/Op18 antigen;
b) treating the dendritic precursor cells with a cytokine to produce dendritic cells stably expressing a stathmin/Op18 antigen;
c) contacting T cells together with the dendritic cells stably expressing the stathmin/Op18 antigen to produce primed T cells; and
d) administering to the patient an effective amount of either the primed T cells, dendritic cells, or a combination thereof.

26. The method of claim 25 wherein the cytokine is selected from the group consisting of interluekins, GM-CSF, TNF, and any combination thereof.

27. The method of claim 26 wherein the patient is human, and wherein the patient has a cancer in any stage of development.

28. The method of claim 27 wherein the cancer is lymphoma, leukemia, myeloma, breast, dermal, oral, colon, penile, vulvar cancer, or any combination thereof.

29. The method of claim 25 wherein the recombinant viral vector is an adeno-associated viral vector.

30. A kit for screening a patient for a cancer, the kit comprising:
a) a probe specific for detection of a stathmin/Op18 sequence.

31. The kit of claim 30 wherein the probe is a single-stranded oligonucleotide sequence, a double-stranded oligonucletide sequence, a polypeptide, or any combination thereof.

32. The kit of claim 31 wherein the patient is human, wherein the cancer is in any stage of development, and wherein the cancer is selected from the group consisting of lymphoma, leukemia, myeloma, breast, dermal, oral, colon, penile, vulvar cancer, and any combination thereof.

33. A composition for treating a patient having a cancer, the composition comprising an effective amount of a stathmin/Op18 sequence.

34. The composition of claim 33 wherein the sequence is selected from the group consisting of single-stranded nucleic acids, double-stranded nucleic acids, polypeptides, and any combination thereof.
